SEQUENCE LISTING (9 sequences)

SEQ ID NO: 1 - DNA, 401 bp, Artificial Sequence
  synthetic
  misc_feature (75)..(75): n = Ala or Gly
  misc_feature (176)..(176): n = Gly or Ala
  misc_feature (250)..(250): n = Thr or Cys
  misc_feature (300)..(300): n = Cys or Gly
acccactgttgagagtcagtggcaagagaagtctcgtcttttacgtcccgtatcaaaatgagtgtacaatacatntctatgtgcgagtgagagcacgggtctgggcctggagagtggaccacctacaattggccatttcggtttgcggaagctgtcaagtcaacgcgagtcctagnatctcatagtcttcgcattaacccgtattaagtggactcgcctacagtttgtcttatgctagcaacccaggcanagtctgtacgcgcgatcgtccactggtagtggggtccttcctggagtgtngtatgggccatccggtctaccttactaacttaggctttaagcgcatttctatgtgcgtgaggtgtcgcattcatacttagtctggtcctaagtctgtcacc

SEQ ID NO: 2 - DNA, 20 bp, Artificial Sequence
  synthetic
tctatgtgcgagtgagagca

SEQ ID NO: 3 - DNA, 20 bp, Artificial Sequence
  synthetic
gcgcgatcgtccactggtag

SEQ ID NO: 4 - DNA, 184 bp, Artificial Sequence
  synthetic
  misc_feature (84)..(84): n = Gly or Ala
  misc_feature (158)..(158): n = Thr or Cys
gcacgggtctgggcctggagagtggaccacctacaattggccatttcggtttgcggaagctgtcaagtcaacgcgagtcctagnatctcatagtcttcgcattaacccgtattaagtggactcgcctacagtttgtcttatgctagcaacccaggcanagtctgtacgcgcgatcgtccactgg

SEQ ID NO: 5 - DNA, 30 bp, Artificial Sequence
  synthetic
gcacgggtctgggcctggagagtggaccac

SEQ ID NO: 6 - DNA, 30 bp, Artificial Sequence
  synthetic
gcatagtctgtacgcgcgatcgtccactgg

SEQ ID NO: 7 - DNA, 30 bp, Artificial Sequence
  synthetic
gtggtccactctccaggcccagacccgtgc

SEQ ID NO: 8 - DNA, 30 bp, Artificial Sequence
  synthetic
ccagtggacgatcgcgcgtacagactatgc

SEQ ID NO: 9 - DNA, 60 bp, Artificial Sequence
  synthetic
gtggtccactctccaggcccagacccgtgcccagtggacgatcgcgcgtacagactatgc

(19) United States
(12) Patent Application Publication
(10) Pub. No.: US 2022/0220542 A1
(43) Pub. Date: Jul. 14, 2022

(54) CAPTURE AND ANALYSIS OF TARGET GENOMIC REGIONS

(71) Applicant: RAPID GENOMICS LLC, GAINESVILLE, FL (US)

(72) Inventor: LEANDRO GOMIDE NEVES, GAINESVILLE, FL (US)

(21) Appl. No.: 17/610,724

(22) PCT Filed: May 13, 2020
(86) PCT No.: PCT/US2020/032615

§ 371 Date: Nov. 12, 2021

Related U.S. Application Data
(60) Provisional application No. 62/846,988, filed on May 13, 2019.

(51) Int. Cl. C12Q 1/6813; C12Q 1/6806

(52) U.S. Cl. CPC C12Q 1/6813; C12Q 1/6806

(57) ABSTRACT

The disclosure pertains to materials and methods for capturing target genomic regions, comprising cleaving the target genomic region using endonucleases at specific recognition sites having a minimum of between about 10 and about 30 nucleotides and capturing the target genomic region by hybridizing to a bridge oligonucleotide. The disclosure also pertains to analyzing the captured target genomic regions. The endonucleases used for cleavage can be programmable endonucleases that specifically bind to the recognition sites to direct the cleavage. The captured target genomic regions can be amplified, preferably, via polymerase chain reaction or rolling circle amplification and detected or sequenced. Further, the invention pertains to kits for performing the methods of the invention, comprising one or more endonucleases designed to cleave one or more target genomic regions and bridge oligonucleotides designed to capture the one or more target genomic regions. The kits can be customized for specific target genomic regions.

### CROSS-REFERENCE TO RELATED APPLICATION

This application claims the benefit of U.S. Provisional Application Ser. No. 62/846,988, filed May 13, 2019, the disclosure of which is hereby incorporated by reference in its entirety, including all figures, tables and amino acid or nucleic acid sequences.

The Sequence Listing for this application is labeled “Seq-List.txt” which was created on May 11, 2020 and is 3 KB. The entire content of the sequence listing is incorporated herein by reference in its entirety.

### BACKGROUND OF THE INVENTION

Capture and analysis, such as sequencing of target genomic regions is extremely important in diverse biotechnological fields ranging from agronomy, taxonomy to medicine.

For example, sequencing target regions in a genome is important in precision and individualized medicine, where understanding the genetic diversity in specific regions of the genome can be linked to phenotypic information about a subject's health, which in turn can dictate treatment options. Also, sequencing target regions of a genome is important in agronomical applications for improving desirable traits in plants, such as seed and food production.

Several methods exist for the analyzing target genomic DNA or RNA sequences. For example, targeted amplification prior to sequencing is common and multiple methods are available to isolate and/or amplify target regions of the genome. As a common theme, the methods known in the art utilize variations of polymerase chain reaction (PCR), ligation-chain reaction or probe hybridization to isolate and amplify target genomic regions. Current methods for isothermal detection of genetic material can be performed, for example, using loop-mediated isothermal amplification (LAMP) or strand displacement amplification. However, these techniques are difficult to optimize for different targets and are not suitable for analyzing long targets. Also, these isothermal techniques are difficult or impossible to be multiplexed for a high number of targets and are not suitable for easily sequencing the amplified products. Characterizing, particularly, detecting and sequencing longer stretches of DNA sequences, for example, at least about 1-50 kilobase (kb), is of particular interest. However, such detection and sequencing are not readily achieved with the conventional methods. Therefore, improved methods for characterizing, such as detecting and sequencing target genomic regions, particularly, detecting and sequencing long stretches of target genomic regions, are desirable.

### BRIEF SUMMARY OF THE INVENTION

Certain embodiments of the invention provide materials and methods for capturing target genomic regions and optionally, further analyzing, such as by detecting and/or sequencing. The materials and methods disclosed herein are particularly suitable for analyzing target genomic regions containing at least about 1 kb, preferably, at least about 10 kb, even more preferably, at least about 30 kb, or most preferably, at least about 50 kb.

According to the methods disclosed herein, target genomic regions can be isolated based on cleavage at two specific recognition sites, each recognition site containing a minimum of between about 10 and about 30 nucleotides. The two recognition sites flank the target genomic region. The cleaved target genomic region can then be captured using an oligonucleotide, referenced herein as a “bridge oligo”. A bridge oligo has sequences at the 3′ and the 5′ ends that hybridize to the sequences at the 3′ and 5′ ends, respectively, of the cleaved target genomic region. The captured target genomic regions can then be analyzed, for example, detected and/or sequenced, such as via amplification and sequencing.

Accordingly, certain embodiments of the invention provide methods for capturing a target genomic region from a genetic material, comprising:

a. cleaving the target genomic region from the genetic material using one or more endonucleases having a first recognition site and a second recognition site, each recognition site comprising a sequence of a minimum of between about 10 and about 30 nucleotides, wherein the recognition sites flank the target genomic region,

b. denaturing the cleaved genetic material into single stranded form, and

c. capturing the target genomic region in the single stranded form by hybridizing the target genomic region to a bridge oligo, the bridge oligo comprising sequences at the 3′ and the 5′ that hybridize to the 3′ and 5′ ends, respectively, of the target genomic region in the single stranded form.

The captured target genomic region can be further analyzed, for example, detected or sequenced. Such analysis can comprise the steps of:

d. ligating the free ends of the single stranded target genomic region hybridized to the bridge oligo to produce a single stranded circular target genomic region that is hybridized to the bridge oligo,

e. optionally, degrading non-circularized genetic material,

f. optionally, amplifying the target genomic region by nucleic acid amplification to produce multiple copies of the target genomic region, and

g. analyzing the amplified target genomic region.

In preferred embodiments, the cleavage of the target genomic region at the recognition sites is performed using a first and a second programmable endonuclease, such as Clustered Regularly Interspaced Short Palindromic Repeats (CRISPR) associated protein 9 endonucleases (Cas9 endonuclease), for example, a first Cas9 endonuclease comprising a first guide RNA (gRNA) having a sequence complementary to the first recognition site and a second Cas9 endonuclease comprising a second gRNA having a sequence complementary to the second recognition site.

In further embodiments, the target genomic region hybridized to the bridge oligo is amplified via an amplification reaction, for example, an isothermal amplification reaction, preferably, via a polymerase chain reaction or rolling circle amplification (RCA) reaction. When only the bridge oligo is used as a primer for amplification, a single stranded amplification product is produced via RCA, which comprises concatenated copies of the target genomic region in single stranded form. One or more primers in addition to the bridge oligo can also be used in an RCA reaction to produce a double stranded amplification product, which comprises concatenated copies of the target genomic region in double stranded form. A conventional PCR reaction can also be used to amplify the target molecules using appropriate primers that bind to the target regions or to a common region introduced by the bridge oligo.

The amplified target genomic region can be detected using techniques known in the art, for example, using a labeled probe complementary to a sequence within the target genomic region. The amplified target genomic region can also be sequenced using techniques known in the art, for example, nanopore sequencing (Oxford Nanopore Technologies™), reversible dye-terminator sequencing (Illumina™) and Single Molecule Real-Time (SMRT) sequencing (PacBio™).

The materials and methods disclosed herein can be modified to capture, and optionally, analyze, multiple target genomic regions, for example, in a multiplex reaction. In such embodiments, multiple pairs of target recognition sites are designed to cleave multiple target genomic regions and these multiple target genomic regions can be captured via a plurality of bridge oligos, each of the plurality of bridge oligos specifically designed to capture a target genomic region. In certain embodiments, the plurality of bridge oligos can be immobilized on a solid substrate, such as a chip. The multiple target genomic regions so captured can be detected or sequenced using techniques known in the art.

Further embodiments of the invention also provide kits for carrying out the methods of the invention. The kits of the invention comprise one or more endonucleases designed to cleave one or more target genomic regions and specific bridge oligos designed to capture the one or more target genomic regions.

In certain embodiments, the kits of the invention comprise one or more guide molecules in the form of DNA or RNA designed to cleave one or more target genomic regions and specific bridge oligos designed to capture the one or more target genomic regions. Such kits can also comprise one or more Cas9 endonucleases.

The kits can further comprise polymerases, ligase, primers and other reagents for amplifying the captured one or more target genomic regions. Even further, the kits can provide instructions to perform the methods of the invention.

### BRIEF DESCRIPTION OF THE DRAWINGS

The patent or application file contains at least one drawing executed in color. Copies of this patent or patent application publication, with color drawing(s), will be provided by the Office upon request and payment of the necessary fee.

FIG. 1. Overview of the methods to capture a target genomic region and two examples of analyzing the target genomic region, namely, detection or sequencing.

FIG. 2. An example of sequencing a captured target genomic region using Single Molecule Real-Time (SMRT) sequencing (PacBio™).

FIG. 3. An example of the detection of multiple target genomic regions using an array of bridge oligos designed to capture multiple target genomic regions.

FIG. 4. An example of amplification of circularized target molecule via PCR. Primers are illustrated containing adapters required for sequencing using reversible dye-terminator sequencing (Illumina™). A) Overview of the process. B) Structure of the bridge oligo needed to capture the sequences and provide landing sites for primers. C) Structure of the primers to allow for simultaneous amplification of the molecule, incorporation of unique sample identifier, and incorporation of the necessary structure for dye-terminator sequencing.

FIG. 5. An example of designing recognition sites and bridge oligos based on two gRNAs that bind to the same strand of a double stranded genome.

FIG. 6. An example of designing recognition sites and bridge oligos based on two gRNAs that bind to the opposite strands of a double stranded genome.

FIG. 7. Another example of designing recognition sites and bridge oligos based on two gRNAs that bind to the opposite strands of a double stranded genome.

FIG. 8. Another example of designing recognition sites and bridge oligos based on two gRNAs that bind to the same strand of a double stranded genome.

FIG. 9. Examples of different approaches to ligate the ends of the target molecules to form a circular single-stranded structure.

FIG. 10. An example of an RCA reaction resulting from a bridge oligo with a non-complementary 5′ end section.

FIG. 11. An example of utilizing a single programmable endonuclease and a primer extending from a target to form a circular single-stranded molecule and a bridge oligo.

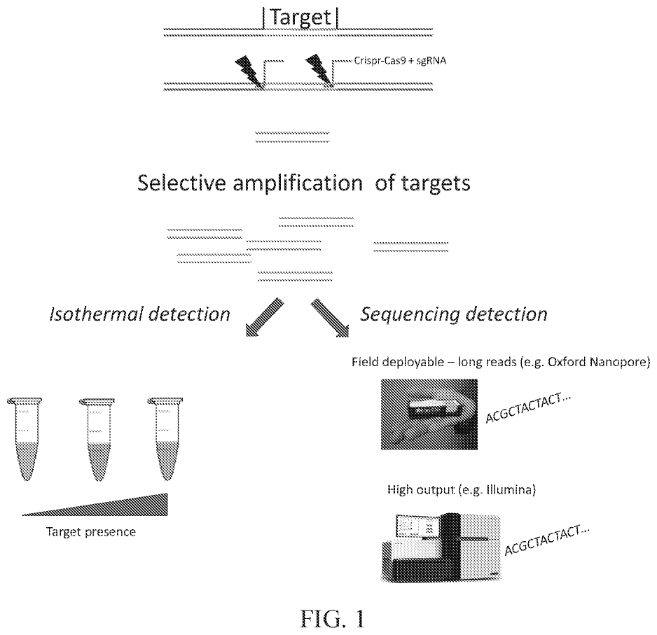

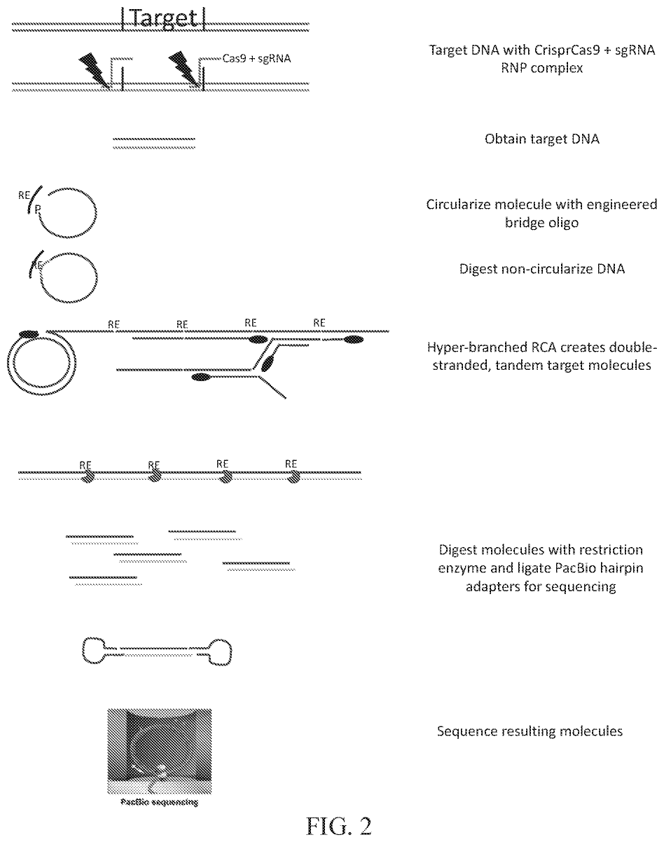

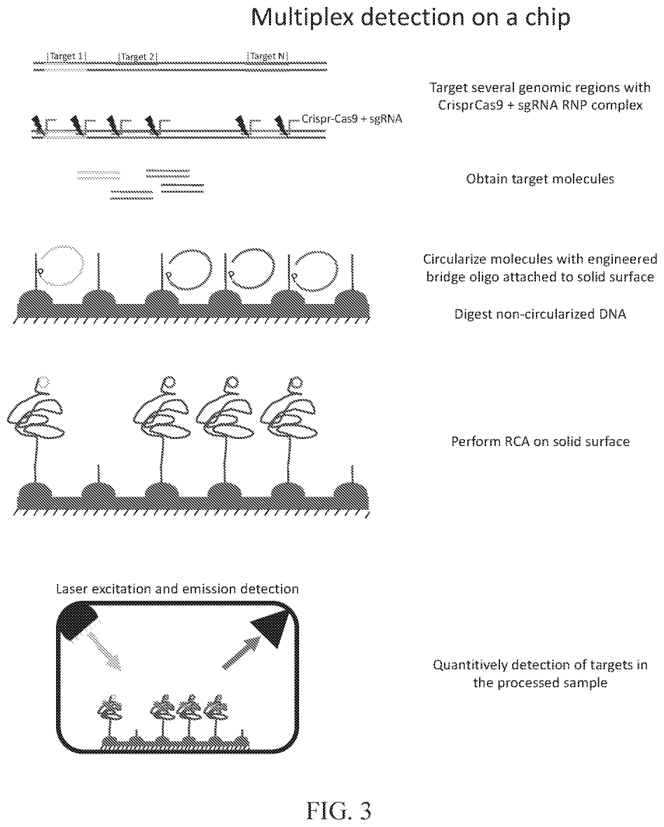

### BRIEF DESCRIPTION OF THE SEQUENCES

SEQ ID NO: 1: Exemplary region to be studied according to the methods of the invention.

SEQ ID NO: 2: Exemplary first recognition site.

SEQ ID NO: 3: Exemplary second recognition site.

SEQ ID NO: 4: Exemplary target genomic region.

SEQ ID NO: 5: Sequence at the 5′ end of the target genomic region of SEQ ID NO: 4.

SEQ ID NO: 6: Sequence at the 3′ end of the target genomic region of SEQ ID NO: 4

SEQ ID NO: 7: Sequence at the 5′ end of the bridge oligo designed to capture target genomic region of SEQ ID NO: 4.

SEQ ID NO: 8: Sequence at the 3′ end of the bridge oligo designed to capture target genomic region of SEQ ID NO: 4.

SEQ ID NO: 9: A bridge oligo designed to capture the target genomic region of SEQ ID NO: 4.

### DETAILED DISCLOSURE OF THE INVENTION

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the described embodiment encompasses embodiments containing the specified materials or steps and those that do not materially affect the basic and novel characteristic(s) of the described embodiment.

The term “about” means within an acceptable error range for the particular value as determined by one of ordinary skill in the art, which will depend in part on how the value is measured or determined, i.e., the limitations of the measurement system. In the context of the lengths of polynucleotides where the terms “about” are used, these polynucleotides contain the stated number of bases or base-pairs with a variation of 0-10% around the value (X±10%).

In the present disclosure, ranges are stated in shorthand, so as to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, such as for the size of the polynucleotides, the combinations and sub-combinations of the ranges (e.g., subranges within the disclosed range) and specific embodiments therein, are explicitly included.

The term “organism” as used herein includes viruses, bacteria, fungi, plants and animals. Additional examples of organisms are known to a person of ordinary skill in the art and such embodiments are within the purview of the invention. The assays described herein can be useful in analyzing any genetic material obtained from any organism.

The term “genome”, “genomic” or “genetic material” and other grammatical variations thereof as used herein refers to genetic material from any organism. A genetic material can be viral genomic DNA or RNA, nuclear genetic material, such as genomic DNA or genetic material present in cell organelles, such as mitochondrial DNA or chloroplast DNA. It can also represent the genetic material coming from a natural or artificial mixture or several organisms.

The phrase “long target genomic regions” used herein refers to a target genomic region having at least about 1 kb, preferably, at least about 10 kb, even more preferably, at least about 30 kb, or most preferably, at least about 50 kb.

The materials and methods disclosed herein for characterizing target genomic regions, particularly, long target genomic regions, solve the problems associated with conventional methods for capture and detection of target genomic regions, particularly, long target genomic regions.

In certain embodiments, the invention provides methods for capturing a target genomic region from a genetic material. The methods comprise the steps of:

a. cleaving the target genomic region from the genetic material using one or more endonucleases having a first recognition site and a second recognition site, each recognition site comprising a sequence of a minimum of between about 10 and about 30 nucleotides, wherein the recognition sites flank the target genomic region,

b. denaturing the cleaved genetic material into single stranded form, and

c. capturing the target genomic region in the single stranded form by hybridizing the target genomic region to a bridge oligo, the bridge oligo comprising sequences at the 3′ and 5′ ends that hybridize to the 3′ and 5′ ends, respectively, of the target genomic region in the single stranded form.

The captured target genomic region can be further analyzed, for example, detected or sequenced. Such analysis can comprise the steps of:

d. ligating the free ends of the single stranded target genomic region hybridized to the bridge oligo to produce a single stranded circular target genomic region that is hybridized to the bridge oligo,

e. optionally, degrading non-circularized genetic material,

f. optionally, amplifying the target genomic region by nucleic acid amplification to produce multiple copies of the target genomic region, and

g. analyzing the amplified target genomic region.

As used herein, “a target genomic region” is a region of interest in the genome of an organism. Such region is flanked by a first recognition site and a second recognition site. Each of the first and the second recognition sites comprises a sequence of a minimum of between about 10 to about 30 nucleotides.

The first and the second recognition sites are selected based on the target genomic region. Typically, unique sequences that flank the target genomic region are selected as the first and the second recognition sites. Uniqueness of these sequences ensures that these sequences are less likely to occur elsewhere in the genome, thus minimizing non-specific cleavage and avoids capture of regions other than the target genomic region. Also, the minimum lengths of the first and the second recognition sites are preferably between about 10 and about 30 nucleotides, which ensure that these sequences occur rarely in a genome, thus also minimizing non-specific cleavage and capture of regions other than the target genomic region. The sequences of the first and the second recognition sites can be identical to each other or different from each other. A person of ordinary skill in the art can determine appropriate sequences for the first and the second recognition sites based on the sequence of the target genomic region and the available genomic sequence for a particular organism, for example, from a genome sequence database.

The cleavage of a genetic material at the recognition sites is performed using one or more endonucleases that cleave the phosphodiester bond (cut) in the genetic material at the specific recognition sites. The endonucleases can be restriction endonucleases. Certain restriction endonucleases that cut recognition sites comprising a minimum of between about 10 and about 30 nucleotides, for example, up to about 80 nucleotides, include meganucleases, such as homing endonucleases from the LAGLIDADG family, GIY-YIG family, HNH family, His-Cys box family and PD-(D/E)XK family. Additional examples of restriction endonucleases that cut recognition sites comprising a minimum of between about 10 and about 30 nucleotides, for example, up to about 80 nucleotides, are known in the art and such embodiments are within the purview of the invention.

In preferred embodiments, cleavage at the recognition sites is performed using one or more programmable endonucleases. The term programmable endonuclease is used to describe different classes of enzymes that can be targeted to cleave a specific region of a DNA or RNA molecule. Thus, a programmable endonuclease is an endonuclease that can be designed or programmed to cleave a nucleotide sequence of interest. For example, a programmable endonuclease can comprise of target recognition portion and endonuclease portion, where a common endonuclease portion can be combined with any target recognition portion to cleave a nucleotide sequence of interest.

In one embodiment, the programmable endonucleases are targeted by a guide RNA (gRNA), a guide DNA (gDNA) or by a structure formed between a guide molecule and the target (Varshney and Burgess, 2016). For example, Cas9 are programmable endonucleases, as they cleave double stranded genetic material by making a double stranded break at a specific location at a recognition site (Jinek et al., 2012). A gRNA having a specific sequence complementary to the sequence of the recognition site directs the Cas9 endonucleases to the recognition site. It has also been shown that gDNA or gRNA molecules can be used to target some types of argonaute proteins (Hegge et al., 2017; Swarts et al., 2015). Additional examples of programmable endonucleases include Cpf1, C2c1, C2c2, C2c, RNA- or DNA-guided Argonaute proteins, structure-guided endonucleases, among others.

Novel proteins can also be engineered to cleave DNA based on recognizing specific DNA structures formed between a gDNA and the target sequence, such as a 3′ end mismatch (Xu et al., 2016). Typically, in the methods of the invention, two Cas9 endonucleases are used to cleave a target genomic region from a genetic material, namely, a first Cas9 endonuclease that cuts DNA at a first recognition site based on a first gRNA having a sequence complementary to the first recognition site and a second Cas9 endonuclease that cuts DNA at a second recognition site based on a second gRNA having a sequence complementary to the second recognition site. The complex containing the gRNA and the components of the Cas9 endonuclease is called the ribonucleoprotein (RNP) complex. In some instances, only one RNP is required to cleave one strand of a double stranded DNA and the selection point on the other strand of the double stranded DNA is a result of a primer-extension reaction (FIG. 11).

The use of programmable endonucleases, such as Cas9, provides significant improvements over other methods known in the art to produce, capture or manipulate circular molecules from target regions (Dahl et al., 2007; Fredriksson et al., 2007). First, the use of common restriction enzymes limits the target genomic regions that can be analyzed because a combination of restriction enzymes might not exist that cut near the target region without cutting within the target region. This is particularly problematic as the number of sites increases. Furthermore, even if such combinations of restriction enzymes exist, they are likely to change every time a new target is envisioned, making the process very difficult to scale. Conversely, using programmable endonucleases allows for virtually any region to be targeted in a scalable process, because the design of unique guide oligos and their synthesis is well-known in the art. Second, the use of PCR instead of endonucleases to extract the sites to be circularized, as done in Fredriksson et al., (2007), limits the size of the target molecules because PCR tends to work best for fragments under 1 Kb. In addition, PCR requires laborious optimization and can be very difficult, cost-prohibitive or impossible to be carried out for a large number of target regions in parallel.

The sequences of the first and the second recognition sites are expressed in this disclosure from 5′ to 3′ direction. Therefore, a sequence toward the 5′ end of the gRNA is complementary to the sequence of the recognition site. In addition to a sequence complementary to a recognition site, a Cas9-gRNA RNP complex also recognizes a short conserved sequence motif of about two to five nucleotides, typically, three nucleotides, located adjacent on the non-complementary strand of the target DNA and to the 3′ end of the sequence of the gRNA that is complementary to the recognition site, which is called protospacer adjacent motif (PAM). PAM is critical in the gRNA binding to the recognition site as well as Cas9 mediated cleavage of the target genetic material, although engineered Cas9 enzymes are envisioned that lack this requirement of a PAM site.

One example of a PAM is the sequence NGG, but different Cas9 enzymes are known to require different PAM sites and enzymes are being modified to tolerate variable PAM sites (Hu et al., 2018). Therefore, in certain embodiments of gRNA useful in the materials and methods disclosed herein, a recognition site is designed so that the sequence of the recognition site on the genetic material is immediately followed toward the 3′ side of the non-complementary strand by the sequence 5′-NGG-3′.

When the gRNA binds to the recognition site, the Cas9 endonuclease creates a double stranded break in the double stranded genetic material at three nucleotides toward the 5′ side of the NGG sequence on the non-complementary strand, i.e., starting from the 5′ end and going towards the 3′ end of the recognition site, Cas9 endonuclease makes a double stranded break between the third and the fourth nucleotide.

Once a genetic material is cleaved using appropriate endonucleases, the resultant mixture of the target genomic regions and the cleaved fragments of the genetic material are denatured to convert it into single stranded form, for example, by subjecting it to denaturation conditions. Typically, subjecting cleaved genetic material to denaturation conditions comprises subjecting it to an appropriate temperature in the presence of appropriate compounds, such as salt, dimethyl sulfoxide, sodium hydroxide, etc. and at appropriate pH. DNA can also be denatured using chemical treatment with NaOH or high salt concentration. In preferred embodiments, the cleaved genetic material is denatured by subjecting it to a temperature of: between 75° C. to 115° C., preferably, between 80° C. to 110° C., more preferably, between 85° C. and 105° C. and even more preferably, between 90° C. and 100° C., and most preferably, about 95° C. A person of ordinary skill in the art can determine appropriate denaturation conditions and such embodiments are within the purview of the invention.

To capture the target genomic region separated from the genetic material, a specifically designed bridge oligo is contacted with the cleaved and denatured genetic material. Typically, the bridge oligo is a single stranded oligonucleotide. The bridge oligo comprises sequences at the 3′ and 5′ ends that hybridize to the sequences at the 3′ and the 5′ ends, respectively, of the single stranded target genomic region. A bridge oligo can also be a double stranded oligonucleotide having 3′ and 5′ end overhangs that hybridize to the sequences at the 3′ and the 5′ ends, respectively, of the single stranded target genomic region, which can be used to capture both strands of the target DNA molecule. The bridge oligos can be protected against nuclease degradation by the different ways known in the art so that they remain present in the reaction after a treatment with given nucleases, such as adding one or more phosphorothioate bond in its 3′ and/or 5′ ends, and adding inverted dT and inverted ddT at its 3′ and 5′ end, respectively.

The bridge oligo serves several purposes. The bridge oligo circularizes the correct target genomic regions generated by on-target cuts with the endonucleases. Because off-target cutting is expected in a reaction with endonucleases, a bridge oligo provides additional specificity by only allowing the correct molecules to be circularized. Also, the bridge oligo itself can serve as the primer for the subsequent amplification.

Further, the bridge oligo can be engineered to provide additional functionality, such as preparing the resulting molecules for sequencing. For example, a bridge oligo can be immobilized on a solid surface for detection on a chip or it can be biotinylated for recovery with streptavidin-bound beads. The 5′ and 3′ end of the bridge oligos can also have additional “tail” sequences that are non-complementary to the target region, creating common sequences that can be used to link to the molecule additional functionalities, such as providing sites for binding of primers for PCR, add biotin molecules or other modifications known in the art (FIG. 10).

Between the sequences at the two ends that hybridize with the target genomic regions, a bridge oligo can further contain sequences, such as a restriction site specific for a rare-cutter restriction endonuclease, a primer binding sequence or a target for a programmable endonuclease. The rare-cutter restriction site or the target for a programmable endonuclease can be used to cleave individual target genomic regions from the concatenated copies of the target genomic region produced after nucleic acid amplification. Non-limiting examples of rare-cutter restriction endonucleases are described in PCT Publication WO 2009/079488, which is herein incorporated by reference in its entirety, particularly, Table 1.

As used herein, “a rare-cutter restriction endonuclease” is an endonuclease whose restriction site occurs rarely in a genetic material. For example, for human genome, a rare-cutter restriction endonuclease is an endonuclease whose restriction site occurs on average every 50-100 kb, preferably, every 100-200 kb, or more preferably, every 200-400 kb, or even more preferably, every 400-600 Kb. Examples of rare-cutter restriction endonucleases for human genome and their restriction sites are given in Table 1 below:

TABLE 1
Examples of human rare-cutter
endonucleases and their restriction sites.

| | | Frequency |
|---|---|---|
| Restriction | Recognition | in Human |
| Enzyme | site | genome (kb) |
| Not I | GCGGCCGC | 1000 |
| Xma III | CGGCCG | 100 |
| Sst II | CCGCGG | 100 |
| Sal I | GTCGAC | 100 |
| Nru I | TCGCGA | 300 |
| Nhe I | GCTAGC | 100 |

Additional rare-cutter endonucleases are described in, e.g., Restriction Endonucleases ((Nucleic Acids and Molecular Biology) by Pingoud (Editor), Springer; 1 ed. (2004)). Many rare-cutter endonucleases are also commercially available, such as roaming class of endonucleases, e.g., from New England BioLabs (Beverly, Mass.). Even further examples of rare-cutter endonucleases are known in the art and such embodiments are within the purview of the invention.

A primer binding sequence in a bridge oligo facilitates using an additional primer in a RCA reaction to amplify the target genomic regions and produce concatenated copies of the target genomic region in double stranded form, through hyper-branching of the original molecule.

The first and the second recognition sites can be present on the same strand of the genetic material or on the opposite strands of the genetic material.

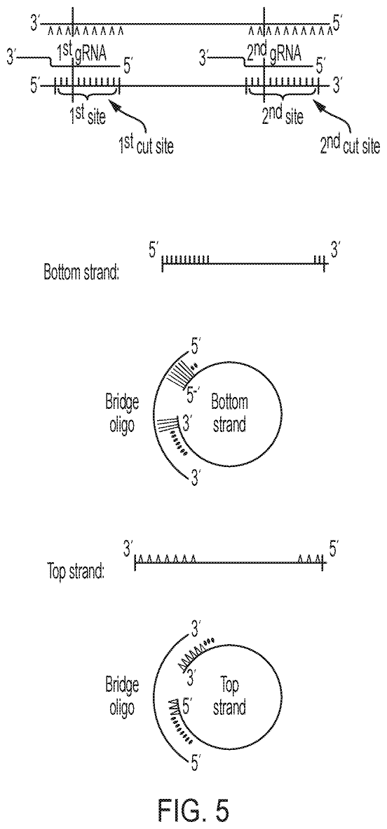

FIG. 5 provides an example of designing recognition sites and bridge oligos based on two gRNAs that bind to the same strand of a double stranded genome. As shown in FIG. 5, the bottom strand of the double stranded genomic DNA is selected for designing the recognition sites. The first gRNA binds to the first recognition site and the Cas9 endonuclease cuts the genomic DNA three nucleotides downstream of the 5′ end of the first recognition site. The second gRNA binds to the second recognition site and the Cas9 endonuclease cuts the genomic DNA three nucleotides downstream of the 5′ end of the second recognition site. Thus, the target genomic region is cleaved from the genomic DNA in double stranded form. This target genomic region has, at the end towards the first recognition site, all but the first three nucleotides from the first recognition site and has, at the end towards the second recognition site, the first three nucleotides from the second recognition site. This double stranded target genomic region can be converted to single stranded form and one or more bridge oligos can be designed to capture either or both of the strands.

To capture the bottom strand from FIG. 5, a bridge oligo is designed to have, towards the 3′ end, a sequence complementary to the first three nucleotides of the second recognition site and additional nucleotides that are complementary to the sequence present on the bottom strand beyond and adjacent to the 5′ end of the second recognition site. This bridge oligo has, towards the 5′ end, a sequence complementary to the first recognition site except the first three nucleotides and optionally, additional nucleotides that are complementary to the sequence present on the bottom strand beyond and adjacent to the 3′ end of the first recognition site.

To capture the top strand from FIG. 5, a bridge oligo is designed to have, towards the 3′ end, the sequence of the first recognition site except the first three nucleotides and optionally, a sequence present on the bottom strand beyond and adjacent to the 3′ end of the first recognition site. This bridge oligo has, towards the 5′ end, the sequence of the first three nucleotides of the second recognition site and additional nucleotides that are present on the bottom strand beyond and adjacent to the 5′ end of the second recognition site.

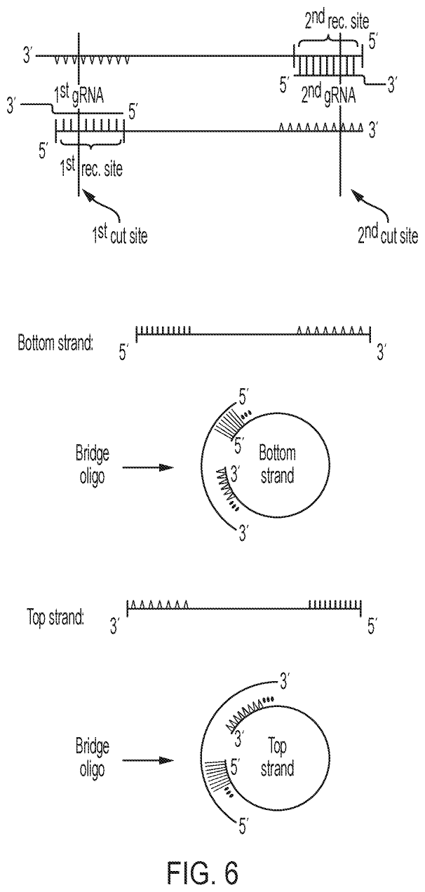

FIG. 6 provides an example of designing recognition sites and bridge oligos based on two gRNAs that bind to the opposite strands of a double stranded genome. As shown in FIG. 6, the bottom strand of the double stranded genomic DNA is selected for designing the first recognition site and the top strand of the double stranded genomic DNA is selected for designing the second recognition site. The first gRNA binds to the first recognition site on the bottom strand and the Cas9 endonuclease cuts the genomic DNA three nucleotides downstream of the 5′ end of the first recognition site. Similarly, the second gRNA binds to the second recognition site on the top strand and the Cas9 endonuclease cuts the genomic DNA three nucleotides downstream of the 5′ end of the second recognition site. Thus, the target genomic region is cleaved from the genomic DNA in double stranded form. This target genomic region has, at the end towards the first recognition site, the sequence of the first recognition site except the first three nucleotides, and has, at the end towards the second recognition site, the sequence of the second recognition site except the first three nucleotides. This double stranded target genomic region can be converted to single stranded form and one or more bridge oligos can be designed to capture either or both of the strands.

To capture the bottom strand from FIG. 6, a bridge oligo is designed to have, towards the 3′ end, the sequence of the second recognition site except the first three nucleotides and optionally, a sequence present on the top strand beyond and adjacent to the 3′ end of the second recognition site. This bridge oligo has, towards the 5′ end, the sequence complementary to the first recognition site except the first three nucleotides and optionally, a sequence complementary to the sequence present on the bottom strand beyond and adjacent to the 3′ end of the first recognition site.

To capture the top strand from FIG. 6, a bridge oligo is designed to have, towards the 3′ end, the sequence of the first recognition site except the first three nucleotides and optionally, a sequence present on the bottom strand beyond and adjacent to the 3′ end of the first recognition site. This bridge oligo has, towards the 5′ end, a sequence complementary to the second recognition site except the first three nucleotides and optionally, a sequence complementary to the sequence present on the top strand beyond and adjacent to the 3′ end of the second recognition site.

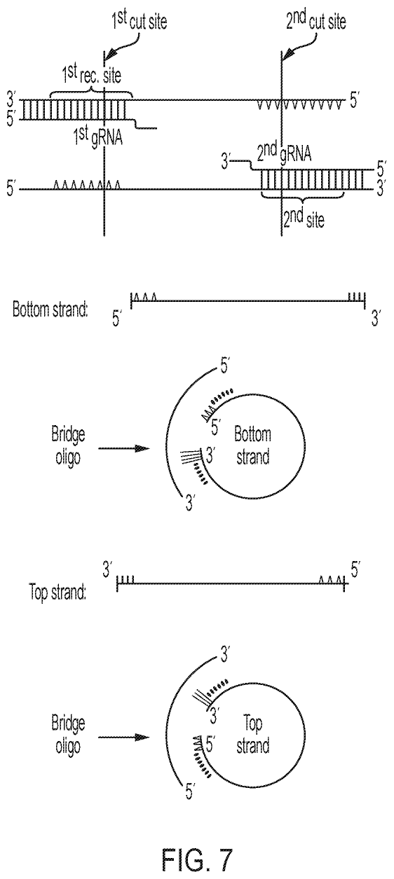

FIG. 7 provides another example of designing recognition sites and bridge oligos based on two gRNAs that bind to the opposite strands of a double stranded genome. As shown in FIG. 7, the top strand of the double stranded genomic DNA is selected for designing the first recognition site and the bottom strand of the double stranded genomic DNA is selected for designing the second recognition site. The first gRNA binds to the first recognition site on the top strand and the Cas9 endonuclease cuts the genomic DNA three nucleotides downstream of the 5′ end of the first recognition site. Similarly, the second gRNA binds to the second recognition site on the bottom strand and the Cas9 endonuclease cuts the genomic DNA three nucleotides downstream of the 5′ end of the second recognition site. Thus, the target genomic region is cleaved from the genomic DNA in double stranded form. This target genomic region has, at the end towards the first recognition site, the first three nucleotides of the first recognition site and has, at the end towards the second recognition site, the first three nucleotides of the second recognition site. This double stranded target genomic region can be converted to single stranded form and one or more bridge oligos can be designed to capture either or both of the strands.

To capture the bottom strand from FIG. 7, a bridge oligo is designed to have, towards the 3′ end, a sequence complementary to the first three nucleotides of the second recognition site and additional nucleotides that are complementary to the sequence present on the bottom strand beyond and adjacent to the 5′ end of the second recognition site. This bridge oligo has, towards the 5′ end, the sequence of the first three nucleotides of the first recognition site and additional nucleotides that are present on the top strand beyond and adjacent to the 5′ end of the first recognition site.

To capture the top strand from FIG. 7, a bridge oligo is designed to have, towards the 3′ end, a sequence complementary to the first three nucleotides of the first recognition site and additional nucleotides that are complementary to the sequence present on the top strand beyond and adjacent to the 5′ end of the first recognition site. This bridge oligo has, towards the 5′ end, the sequence of the first three nucleotides of the second recognition site and additional nucleotides that are present on the bottom strand beyond and adjacent to the 5′ end of the second recognition site.

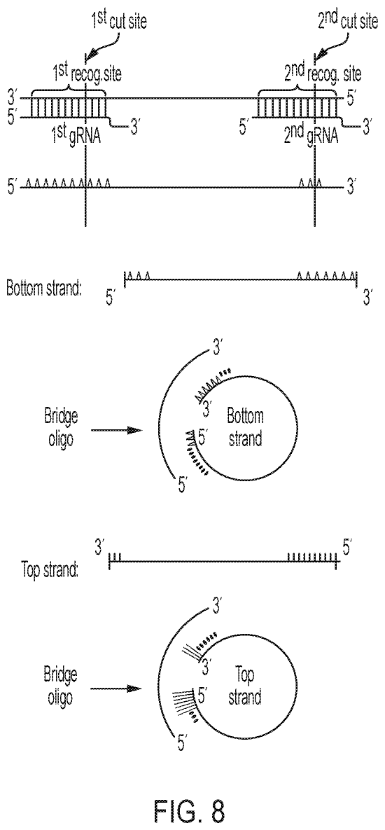

FIG. 8 provides another example of designing recognition sites and bridge oligos based on two gRNAs that bind to the same strand of a double stranded genome. As shown in FIG. 8, the top strand of the double stranded genomic DNA is selected for designing the first recognition site and the second recognition site. The first gRNA binds to the first recognition site on the top strand and the Cas9 endonuclease cuts the genomic DNA three nucleotides downstream of the 5′ end of the first recognition site. Similarly, the second gRNA binds to the second recognition site on the top strand and the Cas9 endonuclease cuts the genomic DNA three nucleotides downstream of the 5′ end of the second recognition site. Thus, the target genomic region is cleaved from the genomic DNA in double stranded form. This target genomic region has, at the end towards the first recognition site, the first three nucleotides at the 5′ end of the first recognition site and has, at the end towards the second recognition site, all but the first three nucleotides at the 5′ end of the second recognition site. This double stranded target genomic region can be converted to single stranded form and one or more bridge oligos can be designed to capture either or both of the strands.

To capture the bottom strand from FIG. 8, a bridge oligo is designed to have, towards the 3′ end, the sequence of the second recognition site except for the first three nucleotides and optionally, the sequence present on the top strand beyond and adjacent to the 3′ end of the second recognition site. This bridge oligo has, towards the 5′ end, the sequence of the first three nucleotides of the first recognition site and additional nucleotides that are present on the top strand beyond and adjacent to the 5′ end of the first recognition site. To capture the top strand from FIG. 8, a bridge oligo is designed to have, towards the 3′ end, a sequence complementary to the first three nucleotides of the first recognition site and additional nucleotides that are complementary to the sequence present on the top strand beyond and adjacent to the 5′ end of the first recognition site. This bridge oligo has, towards the 5′ end, a sequence complementary to the second recognition site except the first three nucleotides and optionally, a sequence complementary to the sequence present on the top strand beyond and adjacent to the 3′ end of the second recognition site.

The sequences of the bridge oligos represented in FIGS. 5-8 and described in the preceding paragraphs do not need to be perfectly complementary or identical to the corresponding sequences as long as the bridge oligo can hybridize with the corresponding sequences on the target genomic regions. Therefore, certain degree of mismatch can be allowed and such variation is within the purview of the invention.

The term “hybridizes with” indicates that the two sequences are sufficiently complementary to each other to allow hybridization between the two sequences. Sequences that hybridize with teach other can be perfectly complementary but can also have mismatches to a certain extent. Therefore, the sequences at the 5′ and 3′ ends of a bridge oligo may have a few mismatches with the corresponding sequence at the 5′ and 3′ ends of the target genomic region as long as the bridge oligo can hybridize with and capture the target genomic region. Depending upon the stringency of hybridization, a mismatch of about 5% to about 20% between the two complementary sequences would allow for hybridization between the two sequences. Typically, high stringency conditions have higher temperature and lower salt concentration and low stringency conditions have lower temperature and higher salt concentration. High stringency conditions for hybridization are preferred, and therefore, the sequences at the 3′ and 5′ ends of a bridge oligo are preferred to be perfectly complementary to the sequences at the 3′ and 5′ ends, respectively, of the target genomic region.

The captured target genomic region can be further analyzed, for example, detected or sequenced. Such analysis can comprise the steps of:

d. ligating the free ends of the single stranded target genomic region hybridized to the bridge oligo to produce a single stranded circular target genomic region that is hybridized to the bridge oligo,

e. optionally, degrading non-circularized genetic material,

f. optionally, amplifying the target genomic region by nucleic acid amplification to produce multiple copies of the target genomic region, and

g. analyzing the amplified target genomic region.

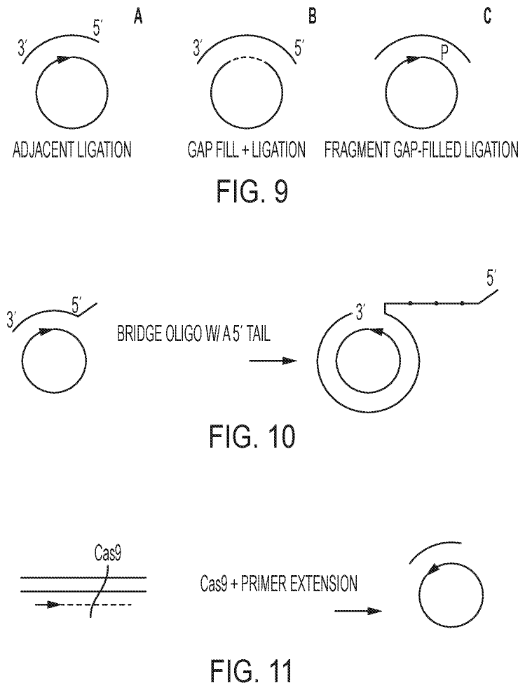

In some embodiments, ligating the free ends of the single stranded target genomic region hybridized to the bridge oligo to produce a single stranded circular target genomic region that is hybridized to the bridge oligo is performed by using a ligase (FIG. 9A). When additional sequences are present in the bridge oligo beyond the sequences complementary to ends of the target genomic region, ligating the free ends of the single stranded target genomic region also comprises nucleic acid synthesis to fill the gap between the two ends of the target genomic region using an appropriate DNA polymerase enzyme (FIG. 9B). The gap between the ends of the target can also be filled by hybridizing a single-stranded oligo to the bridge oligo in the gap and ligating the resulting molecules together (FIG. 9C). Ligase enzymes suitable for ligating the free ends of the single stranded target genomic are known in the art and include T4 DNA ligase, Ampligase, T7 DNA ligase and Taq DNA ligase.

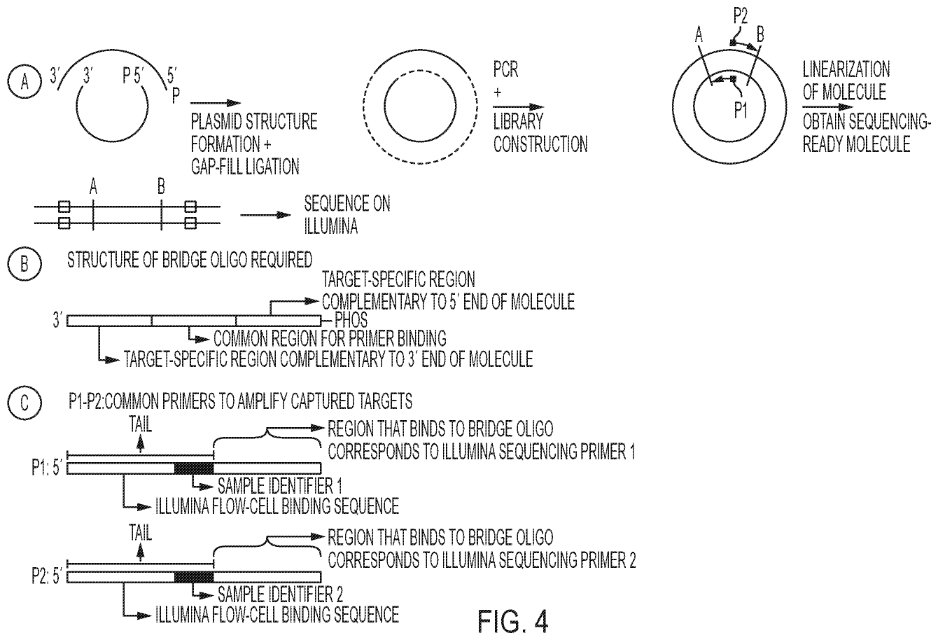

In certain embodiments, the properly circularized target sequences can be converted into a double-stranded vector. This can be done by supplementing the reaction with a DNA polymerase without strand-displacement capabilities and DNA ligase, which will fill synthesize a second strand complementary to the circularized target (FIG. 4).

In certain embodiments, once the target genomic regions are properly circularized, the remaining genetic material, for example, uncleaved genomic DNA and off-targets genomic fragments, are removed, for example, degraded. This can be done by a combination of exonucleases that degrade the nucleic acids from their exposed 3′ or 5′ terminus, whether single-stranded or double-stranded, DNA or RNA. Thus, a treatment with appropriate exonucleases leaves only the circularized target molecules and greatly reduces the difficulty of the subsequent steps.

In certain embodiments, the properly circularized target sequences, separated from the rest of the genetic material via a treatment with an exonuclease and hybridized to the bridge oligo, can be amplified via nucleic acid amplification. In certain preferred embodiments, the amplification can be done via PCR, using the sequences in the bridge oligo to design primers that can amplify the circular molecules and convert them back into linear molecules. This is particularly suitable for short stretches for DNA (<10 Kb), where PCR is most efficient. The bridge oligos for multiple targets can contain a common region non-complementary to the target that is used to drive the PCR reaction with a pair of common primers for all fragments in parallel (FIG. 4). In other preferred embodiments, such amplification is an isothermal amplification, for example, rolling circle amplification (RCA). The isothermal amplification facilitates amplification of long stretches of DNA, for example, multiple copies of target genomic regions, each copy containing at least about 10-50 kb can be produced.

In a conventional RCA reaction, padlock synthetic probes are used to hybridize a target nucleic acid sequence and create a circle (Nilsson et al., 1994). The circle is then amplified in an RCA reaction. In the instant invention, a circularized target sequence captured using a bridge oligo is amplified via an RCA reaction. In such RCA reaction, the bridge oligo acts as the primer for the RCA reaction, which linearly amplifies the target sequence creating long, single-stranded, tandem repeats of the target sequence. The amplified single stranded target sequences can then be used for subsequent analysis. The amplified target sequences can also be converted into double-stranded form, for example by capturing both target strands and allowing their RCA products to reanneal.

In some embodiments, exponential amplification of the target circularized molecules can be obtained through hyper-branched RCA using one or more additional primers (beyond the bridge oligo itself) to generate double-stranded tandem repeats of the target sequence. The primers beyond the bridge oligo can be designed based on the sequences of the bridge oligo, based on the sequences present in the target genomic region, or be composed of a combination of random bases to amplify multiple positions of the circle.

Additional approaches to convert the single stranded captured target genomic regions into double-stranded form are known in the art and such embodiments are within the purview of the instant invention.

The target genomic regions of different sizes can be created and amplified by RCA, thereby bypassing limitations of conventional methods that depend on amplifying nucleic acids by PCR and, hence, are not effective for amplifying long target genomic regions. An important advantage provided by the methods disclosed herein is the detection and analysis of long target genomic regions, for example, genomic regions containing at least about 1 kb, preferably, at least about 15 kb, even more preferably, at least about 30 kb, or most preferably, at least about 50 kb.

In certain embodiments, the product of the RCA reaction can be analyzed to detect or sequence the target genomic region. For example, amplification product can be detected based on the turbidity of the reaction, fluorescence detection or labeled molecular beacons.

The term “label” refers to a molecule detectable by spectroscopic, photochemical, biochemical, immunochemical, chemical, or other physical means. For example, useful labels include fluorescent dyes (fluorophores), fluorescent quenchers, luminescent agents, electron-dense reagents, biotin, digoxigenin, ³²P and other isotopes or other molecules that can be made detectable, e.g., by incorporating into an oligonucleotide. The term includes combinations of labeling agents, e.g., a combination of fluorophores each providing a unique detectable signature, e.g., at a particular wavelength or combination of wavelengths.

Exemplary fluorophores include, but are not limited to, Alexa dyes (e.g., Alexa 350, Alexa 430, Alexa 488, etc.), AMCA, BODIPY 630/650, BODIPY 650/665, BODIPY-FL, BODIPY-R6G, BODIPY-TMR, BODIPY-TRX, Cascade Blue, Cy2, Cy3, Cy5, Cy5.5, Cy7, Cy7.5, Dylight dyes (Dylight405, Dylight488, Dylight549, Dylight550, Dylight 649, Dylight680, Dylight750, Dylight800), 6-FAM, fluorescein, FITC, HEX, 6-JOE, Oregon Green 488, Oregon Green 500, Oregon Green 514, Pacific Blue, REG, Rhodamine Green, Rhodamine Red, ROX, R-Phycoerythrin (R-PE), Starbright Blue Dyes (e.g., Starbright Blue 520, Starbright Blue 700), TAMRA, TET, Tetramethylrhodamine, Texas Red, and TRITC.

Certain embodiments of the invention provide detection in parallel of more than four different targets. For example, different bridge oligos can be immobilized onto a substrate, for example, a chip, where the coordinates of each bridge oligo and, therefore, of each target genomic region is known. Using such substrate, dozens, hundreds or even thousands of target sequences can be detected and analyzed in a multiplex reaction.

In additional embodiments, the product of the PCR or RCA reaction is sequenced. Various methods of sequencing can be used for sequencing the product of the PCR and RCA, such as using portable Nanopore Minion™ or benchtop machines, Nanopore Promethion™, PacBio Sequel™ or Illumina HiSeq™. The sequencing step can also be used for multiplex detection of several targets and/or polymorphism detection.

In certain embodiments, short properly circularized target sequences can be prepared for sequencing after PCR amplification (FIG. 4). The PCR primers can be specific to the target regions or, preferably, be universal to all targets by designing them to amplify a common region introduced by the bridge oligo (FIGS. 4B-4C). Once amplified, adapter molecules can be added to the molecules to make them compatible with the sequencer being used, following the specific manufacture's recommendations. Alternatively, primers with “tails” non-complementary to the bridge oligo can be used to incorporate the necessary adapters during the PCR, making the process more efficient (FIGS. 4A and 4C). These primers may also contain a short unique sequence (4-16 nucleotides) that is added during PCR to link the sequencing data to the respective sample, commonly known as index or barcode (FIG. 4C).

In certain embodiments, concatenated copies can be cleaved to produce individual copies of the target genomic region, for example, using a rare-cutter restriction enzyme to cut the concatenated copies at the restriction site introduced via the bridge oligo. Such cleavage produces multiple copies of the target genomic region, each having sticky ends.

The sticky ends can be used to conjugate the target genomic regions to an adapter sequence. For example, an adapter comprising overhangs complementary to the restriction site specific for the rare-cutter restriction enzyme can be mixed with the copies of the target genomic regions to produce a double stranded DNA comprising the target genomic region flanked by the adapters.

The term “adapter” as used herein refers to a known nucleotide sequence of between four to one hundred nucleotides, preferably, between ten to twenty nucleotides, and even more preferably, about fifteen nucleotides, depending on the sequencing technology being used. The adapter sequences once incorporated at the ends of the amplified copies of the target genomic regions can facilitate sequencing of the target genomic regions, for example, by providing binding sites for primers. In one embodiment, target genomic regions flanked by the adapters are sequenced using paired-end sequencing.

The term “paired-end sequencing” used herein refers to the sequencing technology where both ends of a fragment are sequenced using specific primer binding sites present on each of the ends of the double stranded polynucleotides. Paired-end sequencing generates high-quality sequencing data which is aligned using a computer software program to generate the sequence of the polynucleotide flanked by the two primer binding sites. Sequencing from both ends of a double stranded molecule allows high quality data from both ends of the double stranded molecule because sequencing from only one end of the molecule may cause the sequencing quality to deteriorate as longer sequencing reads are performed.

In the paired-end sequencing, the double stranded polynucleotides produced at the end of the adapter incorporation are sequenced using specific primers that bind to the two ends of the double stranded target genomic regions flanked by the adapters. A general description and the principle of paired-end sequencing is provided in Illumina Sequencing Technology, Illumina, Publication No. 770-2007-002, the contents of which are herein incorporated by reference in their entirety.

Non-limiting examples of the paired-end sequencing technology are provided by Illumina MiSeq™, Illumina MiSegDx™ and Illumina MiSegFGx™. Additional examples of the paired-end sequencing technology that can be used in the assays of the invention are known in the art and such embodiments are within the purview of the invention.

In certain embodiments, the sticky ends of the cleaved copies of target genomic regions can be used to conjugate the target genomic regions with hairpin adapters. For example, a hairpin adapter comprising overhangs complementary to the restriction site specific for the rare-cutter restriction enzyme can be mixed with the copies of the target genomic regions to produce a double stranded DNA comprising the target genomic region flanked by the hairpin adapters.

As used herein, the phrase “hairpin adapter” refers to a polynucleotide containing a double stranded stem and a single stranded hairpin loop. The single stranded hairpin loop region of a hairpin adapter can provide primer binding site for sequencing. Thus, once a hairpin adapter hybridizes with both sticky ends of a target genomic sequence, it produces a double-stranded DNA template containing the target genomic region in the double stranded region capped by hairpin loops at both ends. Such template can be used for sequencing the target genomic region via Single Molecule Real-Time (SMRT) sequencing (PacBio™).

Description and the principle of SMRT sequencing is provided in Pacific Biosciences (2018), Publication No.: BR108-100318, the contents of which are herein incorporated by reference in their entirety.

In further embodiments, nanopore technology is used to sequence the target genomic regions. In certain such embodiments, the copies of target genomic regions are processed to sequence the target genomic regions as described, for example, in Nanopore Technology Brochure, Oxford Nanopore Technologies (2019), and Nanopore Product Brochure, Oxford Nanopore Technologies (2018). The contents of both these brochures are herein incorporated by reference in their entireties.

In certain embodiments, multiple target genomic regions are captured and optionally, further analyzed, such as detected or sequenced. In such embodiments, a pair of gRNAs is designed for each target genomic region. For designing a plurality of gRNAs for a plurality of target genomic regions, the sequences of the gRNAs are selected so that the Cas9 endonuclease for one target genomic region does not disrupt other target genomic regions. Based on the known genomic sequences of the concerned organism, a person of ordinary skill in the art can determine appropriate combinations of recognition sites to specifically cleave and isolate multiple target genomic regions. The gRNAs can also be designed and synthesized with degenerate or wobble bases to allow for hybridization to multiple or uncertain locations. For example, this can be done to compensate for known polymorphisms in the gRNA target site, to add multiple degenerate bases to randomly hybridize to multiple positions of the genome, as well as to design gRNAs that hybridize to an unknown gene that encodes a known amino acid sequence based on the degeneracy of the genetic code. In such cases, the bridge oligo may also be adapted to contain the degenerate bases, if needed.

Accordingly, certain embodiments of the invention provide a method for capturing a plurality of target genomic regions from a genetic material. The methods comprise the steps of:

a. cleaving a plurality of target genomic regions from the genetic material using a plurality of pairs of endonucleases, each pair of endonucleases having a first recognition site and a second recognition site, each recognition site comprising a sequence of a minimum of between about 17 and about 24 nucleotides, wherein each pair of recognition sites flanks a target genomic region from the plurality of target genomic regions,

b. denaturing the cleaved genetic material into single stranded form, and

c. capturing the plurality of target genomic regions in the single stranded form by hybridizing the target genomic regions to a plurality of bridge oligos, wherein each bridge oligo comprises sequences at the 3′ and 5′ ends that hybridize to the 3′ and 5′ ends, respectively, of a target genomic region from the plurality of target genomic regions in single stranded form.

The aspects described above of capturing a target genomic region, for example, designing the recognition sites, endonucleases used in cleaving the genetic material and designing the bridge oligos for capturing the cleaved target genomic regions are also applicable to the instant methods of capturing a plurality of target genomic regions.

In one embodiment of capturing a plurality of target genomic regions, a substrate is provided having a plurality of bridge oligos conjugated to the substrate at specific known locations. A genetic material cleaved with a plurality of pairs of endonucleases is converted into the single stranded form and the plurality of target genomic regions in single stranded form is contacted with the substrate under appropriate conditions to allow hybridization between the bridge oligos conjugated to the substrate the plurality of target genomic regions. Once target genomic regions are hybridized to the corresponding bridge oligos and circularized, the target genomic regions present at specific locations can be further analyzed. In some embodiments, the capture target genomic regions are amplified using RCA and further detected, for example, using laser excitation and emission detection methods.

In certain embodiments, the plurality of target genomic regions are further analyzed, for example, detected or sequenced. Such analysis can comprise the steps of:

d. ligating the free ends of the single stranded target genomic regions hybridized to the plurality of bridge oligos to produce a plurality of single stranded circular genetic materials, each containing a target genomic region from the plurality of target genomic regions, hybridized to the corresponding bridge oligo,

e. optionally, removing non-circularized genetic material from the substrate,

f. optionally, amplifying the plurality of target genomic regions by nucleic acid amplification to produce multiple copies of each target genomic region, and

g. analyzing the amplified plurality of target genomic regions.

The aspects described above of analyzing a target genomic region, for example, ligating the free ends of the single stranded target genomic regions, detecting the target genomic regions or sequencing the target genomic regions are also applicable to the instant methods of analyzing a plurality of target genomic regions.

In certain embodiments, the non-circularized genetic material can be removed by degrading with exonucleases or by washing it from the substrate.

Further embodiments of the invention provide kits for carrying out the assay of the invention. The kits of the invention can contain specific endonucleases necessary to carry out the assay of the invention, specific guide molecules designed to target one or more target genomic regions, a computer software program designed to process the sequencing data obtained from the assay and optionally, materials that provide instructions to perform the assay. In one embodiment, the kit of the invention comprises:

a) one or more pairs of guide molecules,

b) one or more bridge oligos designed to capture one or more target genomic regions.

The kit can further comprise one or more programmable endonucleases.

In addition, the kits can comprise primers for PCR or RCA reaction of circularized target genomic regions, DNA ligase, polymerase and other reagents for PCR or RCA, restriction endonucleases, for example, rare-cutters to cleave concatenated copies of the target genomic regions, and sequencing reagents.

In certain embodiments, the kit of the invention can be customized for one or more specific target genomic regions. For example, a user may provide the sequences of one or more target genomic regions and a kit can be produced to carry out the assay of the invention for the one or more target sequences.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Example 1—Designing Recognition Sites and Capturing a Target Genomic Region

This example describes a typical procedure for capturing and analyzing a target genomic region according to the materials and methods disclosed herein. For example, the following sequence is identified for analysis in this example.

(SEQ ID NO: 1)
5′ACCCACTGTTGAGAGTCAGTGGCAAGAGAAGTCTCGTCTTTTACGTCC
CGTATCAAAATGAGTGTACAATACAT[A/G] [symbol] [symbol]
CGGGTCTGGGCCTGGAGAGTGGACCACCTACAATTGGCCAT
TTCGGTTTGCGGAAGCTGTCAAGTCAACGCGAGTCCTAG[G/A]ATCTCA
TAGTCTTCGCATTAACCCGTATTAAGTGGACTCGCCTACAGTTTGTCTTA
TGCTAGCAACCCAGGCA[T/C]AGTCTGTAC [symbol] [symbol]
TGGGGTCCTTCCTGGAGTGT[C/G]GTATGGGCCATC
CGGTCTACCTTACTAACTTAGGCTTTAAGCGCATTTCTATGTGCGTGAGG
TGTCGCATTCATACTTAGTCTGGTCCTAAGTCTGTCACC3′.

The sequence of SEQ ID NO: 1 provides the sequence of the non-complementary strand, i.e., the gRNA binds to the opposite strand. Hence, the recognition sites are present on the opposite strand and the regions corresponding to the recognition sites are bolded and italicized, NGG PAM motif is underlined and the sites for cleavage by Cas9 endonuclease are indicated by vertical bars. A first gRNA is designed to bind to the first recognition site having the sequence that is reverse complementary to the sequence of TCTATGTGCGAGTGAGAGCA (SEQ ID NO: 2) and the second gRNA is designed to bind to the second recognition site having the sequence that is reverse complementary to the sequence of GCGCGATCGTCCACTGGTAG (SEQ ID NO: 3). The two recognition sites flank a genomic sequence having certain single nucleotide polymorphisms (SNPs) indicated by the nucleotides in the solid brackets. Cas9 mediated cleavage of this sequence will produce a sequence of 184 bp. Sequencing this fragment will provide information about the SNPs present within this region.

Cas9 mediated cleavage of a genetic material containing the sequence of SEQ ID NO: 1 will produce the following fragment, which is the target genomic region.

(SEQ ID NO: 4)
5′GCACGGGTCTGGGCCTGGAGAGTGGACCACCTACAATTGGCCATTTCG
GTTTGCGGAAGCTGTCAAGTCAACGCGAGTCCTAG[G/A]ATCTCATAGT
CTTCGCATTAACCCGTATTAAGTGGACTCGCCTACAGTTTGTCTTATGCT
AGCAACCCAGGCA[T/C]AGTCTGTACGCGCGATCGTCCACTGG3′.

To create a bridge oligo to capture the target genomic region of SEQ ID NO: 4, the following sequences from the ends of the target genomic region are selected, as underlined in the sequence reproduced above:

| (SEQ ID NO: 5) | |
|---|---|
| | 5′ GCACGGGTCTGGGCCTGGAGAGTGGACCAC |
| (SEQ ID NO: 6) | |
| | 5′ GCATAGTCTGTACGCGCGATCGTCCACTGG. |

Sequences complementary to these sequences are produced for the ends of the bridge oligo to capture the target genomic region of SEQ ID NO: 4.

| (SEQ ID NO: 7) | |
|---|---|
| | 5′GTGGTCCACTCTCCAGGCCCAGACCCGTGC3′ |
| (SEQ ID NO: 8) | |
| | 5′CCAGTGGACGATCGCGCGTACAGACTATGC3′ |

SEQ ID NOs: 7 and 8 are connected to create a bridge oligo of the following sequence:

(SEQ ID NO: 9)
5′GTGGTCCACTCTCCAGGCCCAGACCCGTGCCCAGTGGACGATCGCGCG
TACAGACTATGC3′.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

### REFERENCES

Dahl, F., Stenberg, J., Fredriksson, S., Welch, K., Zhang, M., Nilsson, M., et al. (2007). Multigene amplification and massively parallel sequencing for cancer mutation discovery. Proc. Natl. Acad. Sci. U.S.A 104, 9387-92. doi:10.1073/pnas.0702165104.
Fredriksson, S., Bailer, J., Dahl, F., Chu, A., Ji, H., Welch, K., et al. (2007). Multiplex amplification of all coding sequences within 10 cancer genes by Gene-Collector. Nucleic Acids Res. 35, e47. doi:10.1093/nar/gkm078.
Hegge, J. W., Swarts, D. C., and van der Oost, J. (2017). Prokaryotic Argonaute proteins: novel genome-editing tools? Nat. Rev. Microbiol. 16, 5-11. doi:10.1038/nrmicro.2017.73.
Hu, J. H., Miller, S. M., Geurts, M. H., Tang, W., Chen, L., Sun, N., et al. (2018). Evolved Cas9 variants with broad PAM compatibility and high DNA specificity. Nature 556, 57-63. doi:10.1038/nature26155.
Jinek, M., Chylinski, K., Fonfara, I., Hauer, M., Doudna, J. A., and Charpentier, E. (2012). A programmable dual-RNA-guided DNA endonuclease in adaptive bacterial immunity. Science 337, 816-21. doi:10.1126/science.1225829.
Nilsson, M., Malmgren, H., Samiotaki, M., Kwiatkowski, M., Chowdhary, B. P., and Landegren, U. (1994). Padlock probes: circularizing oligonucleotides for localized DNA detection. Science 265, 2085-8. Available at: http://www.ncbi.nlm.nih.gov/pubmed/7522346 [Accessed May 1, 2018].
Swarts, D. C., Hegge, J. W., Hinojo, I., Shiimori, M., Ellis, M. A., Dumrongkulraksa, J., et al. (2015). Argonaute of the archaeon Pyrococcus furiosus is a DNA-guided nuclease that targets cognate DNA. Nucleic Acids Res. 43, 5120-5129. doi:10.1093/nar/gkv415.
Varshney, G. K., and Burgess, S. M. (2016). DNA-guided genome editing using structure-guided endonucleases. Genome Biol. 17, 187. doi:10.1186/s13059-016-1055-4.
Xu, S., Cao, S., Zou, B., Yue, Y., Gu, C., Chen, X., et al. (2016). An alternative novel tool for DNA editing without target sequence limitation: the structure-guided nuclease. Genome Biol. 17, 186. doi:10.1186/s13059-016-1038-5.

1-45. (canceled)

46. A method for capturing a target genomic region from a genetic material, the method comprising the steps of:
a) cleaving the target genomic region from the genetic material using one or more endonucleases having a first recognition site and a second recognition site, each recognition site comprising a sequence of a minimum of between about 10 and about 30 nucleotides, wherein the recognition sites flank the target genomic region,
b) denaturing the cleaved genetic material into single stranded form, and
c) capturing the target genomic region in the single stranded form by hybridizing the target genomic region to a bridge oligo, the bridge oligo comprising sequences at the 3′ and 5′ ends that hybridize to the 3′ and 5′ ends, respectively, of the target genomic region in the single stranded form.

47. The method of claim 46, wherein each of the first recognition site and the second recognition site comprises a sequence of a minimum of between about 10 to about 30 nucleotides and the first and the second recognition sites flank the target genomic region.

48. The method of claim 46, wherein the one or more endonucleases that cleave the genetic material at the specific recognition sites are restriction endonucleases, meganucleases, or programmable endonucleases.

49. The method of claim 48, wherein the one or more programmable endonucleases are selected from Clustered Regularly Interspaced Short Palindromic Repeats (CRISPR) associated protein 9 endonucleases (Cas9 endonucleases), Cpf1, C2c1, C2c2, C2c, RNA- or DNA-guided Argonaute proteins and structure-guided endonucleases.

50. The method of claim 49, wherein the programmable endonucleases comprise a first endonuclease that cuts DNA at the first recognition site based on a first guide molecule having a sequence complementary to the first recognition site and a second programmable endonuclease that cuts DNA at the second recognition site based on a second guide molecule having a sequence complementary to the second recognition site.

51. The method of claim 46, wherein the bridge oligo is a single stranded oligonucleotide comprising sequences at the 3′ and 5′ ends that hybridize to the sequences at the 3′ and the 5′ ends, respectively, of the single stranded target genomic region.

52. The method of claim 46, wherein the bridge oligo is a double stranded oligonucleotide having 3′ and 5′ end overhangs that hybridize to the sequences at the 3′ and the 5′ ends, respectively, of the single stranded target genomic region.

53. The method of claim 51, wherein the bridge oligo further comprises restriction sites specific for rare-cutter restriction endonucleases, cleavage sites for programmable endonucleases, and/or primer binding sequences.

54. The method of claim 51, wherein the bridge oligo is immobilized on a solid substrate or is biotinylated.

55. The method of claim 46, further comprising analyzing the target genomic region, comprising:
d) ligating the free ends of the single stranded target genomic region hybridized to the bridge oligo to produce a single stranded circular target genomic region that is hybridized to the bridge oligo,
e) optionally, degrading non-circularized genetic material,
f) optionally, amplifying the target genomic region by nucleic acid amplification to produce multiple copies of the target genomic region, and
g) analyzing the amplified target genomic region.

56. The method of claim 55, comprising amplifying the target genomic region by a rolling circle amplification (RCA) reaction to produce multiple concatenated copies of the target genomic region.

57. The method of claim 55, wherein said analyzing comprises sequencing the target genomic region.

58. The method of claim 57, wherein the sequencing comprises nanopore sequencing, reversible dye-terminator sequencing or Single Molecule Real-Time (SMRT) sequencing.

59. A method for capturing a plurality of target genomic regions from a genetic material, comprising:
a) cleaving a plurality of target genomic regions from the genetic material using a plurality of pairs of endonucleases, each pair of endonucleases having a first recognition site and a second recognition site, each recognition site comprising a sequence of a minimum of between about 17 and about 24 nucleotides, wherein each pair of recognition sites flanks a target genomic region from the plurality of target genomic regions,
b) denaturing the cleaved genetic material to single stranded form, and
c) capturing the plurality of target genomic regions in the single stranded form by hybridizing the plurality of target genomic region to a plurality of bridge oligos, wherein each bridge oligo comprises sequences at the 3′ and 5′ ends that hybridize to the 3′ and 5′ ends, respectively, of a target genomic region from the plurality of target genomic regions in single stranded form.

60. The method of claim 59, further comprising analyzing the plurality of target genomic regions, comprising:
d) ligating the free ends of the single stranded target genomic regions hybridized to the plurality of bridge oligos to produce a plurality of single stranded circular target genomic regions, each single stranded circular target genomic region containing a target genomic region from the plurality of target genomic regions hybridized to the corresponding bridge oligo,
e) optionally, removing non-circularized genetic material,
f) optionally, amplifying the plurality of target genomic regions by nucleic acid amplification to produce multiple copies of each target genomic region, and
g) analyzing the amplified target genomic regions.

61. The method of claim 59, wherein the plurality of bridge oligos is immobilized onto a solid substrate.

62. The method of claim 61, comprising amplifying the plurality of target genomic regions by nucleic acid amplification to produce multiple copies of each target genomic region.

63. The method of claim 59, comprising amplifying the target genomic region by polymerase chain reaction (PCR) to produce multiple copies of the target genomic region.

64. The method of claim 63, wherein the PCR primers can be specific to the target regions or universal to all targets by designing them to bind to a common region introduced by the bridge oligo.

65. A kit comprising:
a) one or more guide molecules to direct the endonucleases to the target,
b) one or more bridge oligos designed to capture one or more target genomic regions, and optionally
c) one or more programmable endonucleases.
